(19) United States
(12) Patent Application Publication
(10) Pub. No.: US 2005/0090649 A1
(43) Pub. Date: Apr. 28, 2005

(54) Pseudo-metalloproteins, their preparation and use

(76) Inventors: Angelina Lombardi, Napoli (IT); Vincenzo Pavone, Napoli (IT)

Correspondence Address: Jeffrey A Schwab Jay S Cinamon Thomas E Spath;Abelman, Frayne & Schwab, 150 East 42nd Street, New York, NY, 10017, US

(21) Appl. No.: 10/344,329

(22) PCT Filed: Aug. 9, 2001
(86) PCT No.: PCT/IB01/01427

(30) Foreign Application Priority Data
Aug. 10, 2000 (IT) RM2000A000454

(51) Int. Cl. A61K 38/42; C07K 14/47

(52) U.S. Cl. 530/400

(57) ABSTRACT

Described herein is a compound of general formula (I) in which: M is a metal selected among Fe, Mn, Ti, Mo, Co, Ni, Cu, Pd, Pt, Au, Ru, Cr, V, Tb, Yb, Rh, Ir, Os; X1 is an antigen, or else a functional group that enables association to a biomolecule; X2 is a functional group that enables association to an electrode; S1 and S2 are spacer groups made up of a chain of from 3 to 12 atoms of C, N, O, S and corresponding mixtures; all the other substituents have an amino acid nature. The said compound may be used to construct biosensors.

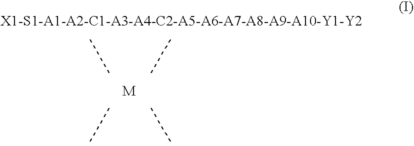

### FIELD OF THE INVENTION

The present invention relates to synthetic metalloproteins, i.e., peptide-based compounds of synthesis, complexed with metal ions, capable of bringing about association, either covalent or non-covalent, with biomolecules such as antibodies, antigens, enzymes, receptors, nucleic acids, peptides and proteins. The invention also regards their preparation and al their use as biosensors, in combination with appropriate electrodes in voltmetric or amperometric measurements.

### PRIOR ART

Synthetic metalloproteins, which form the subject of the present invention, are synthetic models of complex natural systems [Cunningham, B. C., & Wells J. A. (1997) “Minimized Proteins” Curr. Opin. Struct. Biol. 7, 457, DeGrado; W. F., Summa, C. M., Pavone, V., Nastri, F., & Lombardi, A. (1999) “De Novo Design and Structural Characterization of Proteins and Metalloproteins” Annu. Rev. Biochem. 68, 779]. They are of a peptide nature and have dimensions intermediate between models of low molecular weight, containing ligands generally of an organic nature, and the natural proteins and their mutants, previously used in many fields [DeGrado, W. F., Summa, C. M., Pavone, V., Nastri, F., & Lombardi, A. (1999) “De Novo Design and Structural Characterization of Proteins and Metalloproteins” Annu. Rev. Biochem. 68, 779; Nastri F., Lombardi A., D'Andrea L. D., Sanseverino M., Maglio O., and Pavone V. (1998) “Miniaturized hemoproteins”, Biopolymers, 47, 5; Lombardi A., Summa C., Geremia S., Randaccio L., Pavone V. and DeGrado W. F (2000) “Retrostructural Analysis of Metalloproteins; Application to the Design of a Minimal Model for Diiron Proteins”, Proc. Natl. Acad. Sci., 97, 6298]. The synthetic metalloproteins that form the subject of the present invention present such a peculiarity of behaviour to make them extremely competitive as compared to other products appearing in the literature, for the reasons described in what follows [DeGrado, W. F., Summa, C. M., Pavone, V., Nastri, F., & Lombardi, A. (1999) Annu. Rev. Biochem. 68, 779; Nastri F., Lombardi A., D'Andrea L. D., Sanseverino M., Maglio O., and Pavone V. (1998) “Miniaturized hemoproteins”, Biopolymers, 47, 5]. They are in fact simpler systems than their natural counterparts (metalloproteins), but at the same time have a sufficient size and chemical diversity to enable the construction of appropriate functional sits. In particular, the molecules that form the subject of the present invention are particularly suited for making biosensors.

GB-A-2191200 refers to a process for preparing conjugates of biologically active metalloproteins (in particular conjugates between metalloproteins, human albumin and immunoglobulin of IgG type), useful for targeting the metalloprotein to a specific tissue or organ for therapeutic purposes. No biosensor application is claimed.

EP-A-09492690 refers to a biosensor protein, which comprises a fusion protein of a protein of interest containing a hydrophobic region and the chromophore part of the GFP protein, wherein the chromophore part of the GFP protein is located within the hydrophobic region of the protein of interest. The detection of ligand binding gas conformational change or change of the optical properties of the chromophore.

WO 98 53849A describes synthetic metalloproteins, which significantly differ from the synthetic metalloproteins object of the present invention, both in size and in design and synthetic methodologies used. In particular WO 98 53849A relates to natural proteins of known structure (a physiological plasma protein, thioredoxin or human serum albumin), which are synthetically modified to incorporate a redox-active metal site. Applications include catalysis, such as hydrocarbon oxidation and superoxide dismutation reactions. No biosensor application is claimed.

As regards biosensors, those of affinity are analytical instruments that use elements of recognition (for example, antigens, receptor agonists or antagonists) interfaced to a signal transducer which measures the binding of these elements to their specific targets (the analyte). The elements of recognition may be interfaced to different types of transducers of the signal (as described herein), provided that the mechanism of transduction is effective and the corresponding signal can be easily detected. [Turner A. P. F. et al., 1987, Biosensors: Fundamentals and applications, Oxford University Press, N.Y.; Marco M. P. et al., 1995, Immunochemical techniques for environmental analysis: Immunosensors, Trends Anal. Chem. 14, 341; Morgan C. L. et al., 1996, Immunosensors: technology and opportunities in laboratory medicine. Clin. Chem. 42, 193; Skladal P., 1997, Advances in electrochemical immunosensors. Electroanalysis, 9, 737; Rogers K. R. and Mulchandani A., 1998, Affinity Biosensors: Techniques and Protocols, 3, in Methods in Biotechnology, Humana Press, Totowa N.J.].

Up to the present day, different biosensors of affinity have been described (as emerges from the literature cited above), but all of these present considerable applicational limitations. The main elements of a biosensor are the element of recognition and the transducer. In these, the element of recognition is generally a biological macromolecule, such as an antibody or a receptor, which recognizes specific analytes, such as antigens, toxins, drugs, hormones, pesticides and so forth. On account of the stoichiometric relation between elements of recognition and analytes, and of the finite surface area of the transducer of the signal, both the immobilization and the orientation of the element of recognition are critical, in the construction of such biosensors, in order to enable an adequate accessibility of the recognition site for the analyte [Taylor R. F. (1996) Immobilization Methods, pp. 203-219, in Handbook of Chemical and Biological Sensors, IOP, Philadelphia Pa.]. For this purpose reported in the literature is a wide variety of methods, which include: covalent bonding, trapping, “cross-linking”, adsorption. For instance, proteins are utilized that are capable of binding in a specific way particular analytes, such as the protein A, the avidin-biotin system [Nakanishi K. et al., 1996, A novel method of immobilizing antibodies in a quartz crystal microbalance using plasma-polymerized films of immunosensors, Anal. Chem. 68, 1695], terminal thiol groups, and so forth. Elements of recognition have also been trapped in materials, such as polyacrylamide, polyvinyl alcohol, epoxy gel among others [Bhatia S. K. et al., 1989, Anal. Biochem. 178, 408]. Finally, also reported in the literature are bifunctional elements of recognition which present new arrangements of metallic complexes of proteins and peptides on surfaces of gold [Shanzer A. et al. 1999, Functional Monolayers with Coordinatively Embedded Metalloporphyrins, Angew. Chem., 38, 1257; Vogel H. et al., 1999, Functional Molecular Thin Films: Topological Template for the Chemioselective Ligation of Antigens Peptides to Self-Assembled Monolayers, Angew. Chem., 38, 696].

Two methods are known for detecting the phenomenon of the analyte by the biosensor of affinity: the direct method and the indirect method. These are strictly dependent upon the type of transducer used. The former method involves the detection of the analyte by means of the use of a marked analyte as tracer, whereas the latter method may involve both the use of a tracer, detectable by means of optical or electrochemical methods, and the combined use of an enzyme that converts catalytically a substrate into a product which is subsequently detected by the transducer.

Different types of transducers of the signal have been interfaced with the elements of recognition. These envisage mechanisms of transduction of an optical type (fluorescence, bioluminescence), electrochemical type (potenziometric, amperometric, conductimetric), or acoustic type (quartz microbalance). More recently micro-biosensors have also been developed, which make use of electrodes based upon silicon technology [Uhlig A., et al., 1997, Miniaturized ion-selective chip electrode for sensor application, Anal. Chem., 69, 4032; Hintsche R. et al., 1997, Microbiosensor using electrodes made in Si-technology, EXS, 80, 30 267; Scheller F. W., et al., 1991, Second generation biosensors, 6, 245; Paeschke M., et al., 1996, Voltmetric Multichannel Measurement Using Silicon Fabricated Microelectrode Arrays, 8, 891]. The latter technology involves a cascading of events which markedly limits its use, in so far as it requires the biosensor to be made purposely for the specific analyte, the particular event of recognition, the possible use of mediators, or for the enzymatic amplification.

From this reference panorama there emerges the need to develop new biosensors capable of overcoming the limitations encountered in the ones currently available and referred to in what follows: 1) low levels of sensitivity linked to the indirect methods of detection, which involve a large number of manipulations; 2) difficulty of automation in the indirect methods of detection; 3) availability of the tracer itself. In addition, the use of direct methods of detection is markedly limited by the need to orient the element of bioaffinity, which is frequently not effective in transferring the information of the recognition to the electrode.

The synthetic metalloproteins of the present invention enable the problems indicated above to be solved with the major advantage of enabling direct electrochemical detection.

### SUMMARY OF THE INVENTION

An object of the present invention is the creation of synthetic metalloproteins according to formula (I) indicated herein.

Another object is the method of preparation of said synthetic metalloproteins.

Yet another object is the use of said synthetic metalloproteins as biosensors.

A further object is the creation of biosensors with the synthetic metalloproteins according to formula (I) indicated herein, which among other things, enable direct electrochemical detection.

Yet a further object is the use of said biosensors for the following applications: in vitro diagnostics, immunodiagnostics, determination of pollutant substances in water, determination of preservatives in foodstuffs, determination of drugs and/or toxic products in biological fluids, determination of the metabolic pathways of drugs, such as electrochemical probes of biomolecules.

Other objects will emerge clearly from the following detailed description of the invention.

### DETAILED DESCRIPTION OF THE INVENTION

A complete list of the abbreviations and symbols used in the text is provided at the end of the present description.

In the present invention molecules are described that are represented by the following general formula (I): 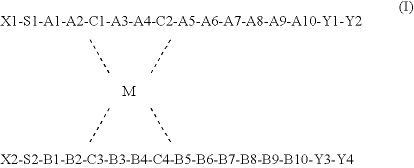
in which:
M is a metal selected in the group made up of Fe, Mn, Ti, Mo, Co, Ni, Cu, Pd, Pt, Au, Ru, Cr, V, Tb, Yb, Rh, Ir, Os, in any of the possible states of oxidation;
X1 is an antigen, or any functional group that enables the association, either covalent or non-covalent, to a biomolecule, by “biomolecule” being meant a molecule of biological interest, such as antibodies, antigens, enzymes, receptors, nucleic acids, peptides and proteins;
X2 is any functional group that enables the association, either covalent or non-covalent, to an electrode;
S1 and S2, which are the same as or different from one another, are spacer groups which, in the main chain, are made up of 3 to 12 atoms chosen in the group of the chemical species C, N, O, S and corresponding mixtures;
C1, C2, C3, C4, which are the same as or different from one another, are natural or synthetic amino acids capable of coordinating the metal ion M;
A1, A2, A9, B1, B2, B9, which are the same as or different from one another, are any natural or synthetic hydrophilic amino acid;
A3, A4, A7, A8, A10, B3, B4, B7, B8, B10, which are the same as or different from one another, are any natural or synthetic amino acid;
A5 and B5, which are the same as or different from one another, are Gly or else any amino acid of configuration D on the alpha carbon;
A6 and B6, which are the same as or different from one another, are any amino acid selected in the group made up of Ala, Abu, Val, Ile, allo-Ile;
Y1 is any natural or synthetic hydrophobic amino acid, and Y2 is Gly, or else Asp, or else Glu, Y3 is any natural or synthetic basic amino acid, and Y4 is NH2; or else Y1 is any natural or synthetic basic amino acid, and Y2 is NH2, Y3 is any natural or synthetic hydrophobic amino acid, and Y4 is Gly, or else Asp, or else Glu; or else Y1 and Y3, which are the same as or different from one another, are any natural or synthetic hydrophobic amino acid, and Y2 and Y4 are NH2.

Amongst the compounds of formula (I) as indicated above particularly preferred are those in which:
M is a metal selected in the group made up of Fe, Mn, Co, Ni, Cu in any of the possible states of oxidation;
X1 is an antigen, or else any functional group that enables the association, either covalent or non-covalent, to a biomolecule, such as amine, sulphidryl, hydroxyl, and biotin; X1 in particular may be a receptor agonist or antagonist, an amino acid functionalized with biotin, an enzymatic inhibitor, an oligonucleotide, or a peptide nucleic acid (PNA).

X2 is any functional group, such as sulphidryl and hydroxyl, that enables the association, either covalent or non-covalent, to an electrode;
S1 and S2, which are the same as or different from one another, are (Gly)n with n=1-4;
C1, C2, C3, C4, which are the same as or different from one another, are amino acids selected amongst natural or synthetic ones which contain on the side chain a functional group, such as amine, carboxyl, hydroxyl, thiol, thioether, imidazole, or pyridyl, which are capable of coordinating the metal ion M;
A1, A2, B1, B2, which are the same as or different from one another, are any amino acid selected in the group made up of Gln, Asn, Thr, allo-Thr, Ser;
A3 and B3, which are the same as or different from one another, are any amino acid selected in the group made up of Ser, Thr, Val, Pro, Ile, allo-Thr, Allo-Ile;
A4 and B4, which are the same as or different from one another, are an amino acid selected in the group made up of Ser, Thr, Val, Pro, Ile, allo-Thr, allo-Ile, Lys;
A5 and B5, which are the same as or different from one another, are Gly, or else any amino acid of configuration D on the alpha carbon;
A6 and B6, which are the same as or different from one another, are any amino acid selected in the group made up of Ala, Abu, Val, Ile, allo-Ile;
A7, A10, B7, B10, which are the same as or different from one another, are any natural or synthetic amino acid;
A8 and B8, which are the same as or different from one another, are any C-alpha,alpha-dialkylated amino acid;
A9 and B9, which are the same as or different from one another, are any natural or synthetic hydrophilic amino acid;
Y1 is any natural or synthetic hydrophobic amino acid, and Y2 is Gly, or else Asp, or else Glu, Y3 is any natural or synthetic basic amino acid, and Y4 is NH2; or else Y1 is any natural or synthetic basic amino acid, and Y2 is NH2, Y3 is any natural or synthetic hydrophobic amino acid, and Y4 is Gly, or else Asp, or else Glu; or else Y1 and Y3, which are the same as or different from one another, are any natural or synthetic hydrophobic amino acid, and Y2 and Y4 are NH2.

By “amino acid” is meant one of the organic compounds containing at least one amine group and one carboxyl group. According to the position of the amine group with respect to the carboxyl group, alpha-amino acids, beta-amino acids, gamma-amino acids, etc., are distinguished.

The term “any amino acid” here used refers to the L and D isomers both of natural amino acids and of “non-proteic” amino acids (also referred to as “synthetic amino acids”) which are commonly used in the chemistry of peptides for the preparation of synthetic analogues of natural peptides. Alpha-amino acids, whether substituted or non-substituted in the alpha and beta positions both of the L configuration and of the D configuration are indicated among the non-proteic amino acids.

The natural amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, gamma-carboxyglutamic acid, arginine, ornithine, and lysine.

Examples of “non-proteic” amino acids are norleucine, norvaline, alloisoleucine, allothreonine homoarginine, thioproline, dehydroproline, hydroxyproline, pipecolic acid, azetidinic acid, homoserine, cyclohexylglycine, alpha-amino-n-butyrric acid, cyclohexylalanine, aminophenylbutyrric acid, beta-(1- or 2-naphthyl) alanine, O-alkylated derivates of serine, threonine and tyrosine, S-alkylated cysteine, S-alkylated homocysteine, epsilon-alkylated lysine, delta-alkylated ornithine.

Examples of C-alpha,alpha-dialkylated amino acids are: alpha,alpha-dimethylglycine, alpha-aminocyclopropane carboxylic acid, alpha-aminocyclobutane carboxylic acid, alpha-aminocyclopentane carboxylic acid, alpha-aminocyclohexane carboxylic acid, diethylglycine, dipropylglycine, diphenylglycine. Other non-proteic amino acids are those reported in: “Diversity of synthetic peptides”, Konishi et al., First International Peptide Symposium, Kyoto, Japan, 1997.

Examples of hydrophilic amino acids are: Glu, Asp, Asn, Gln, Thr, allo-Thr, h-Ser, Ser, Lys, Arg, His, Orn, Dab, Dap.

Examples of hydrophobic amino acids are: Ala, Val, Leu, Ile, allo-Ile, Met, Phe, Tyr, Trp, Cha, Chg, Abu, n-Val, n-Leu, beta-2-Nal.

Examples of basic amino acids are: Lys, Arg, His, Orn, Dab, Dap.

Examples of amino acids capable of coordinating metal ions are: Cys, His, Met, Asp, Glu, Lys, Ser, Thr, h-Cys, h-Ser, Dap, Dab, Orn, Gaba.

The compounds of the general formula (I) may also be used in combination with appropriate counter-ions, provided that they are compatible with the specific applications.

The completely unexpected and unique properties, described in what follows, of the synthetic metalloproteins of the present invention derive from the structural solutions chosen, such as their low molecular weight, and well-defined secondary and tertiary structures.

The compounds of the invention are easy to synthesize and purify and, since they are of low molecular weight, may be obtained on a large scale at lower costs than metalloproteins obtained as products of expression or extraction. The synthetic procedure proposed is based mainly on consolidated methods of solid-phase peptide synthesis, preferably synthesis with protector groups characteristic of Fmoc chemistry [Atherton, E. & Sheppard, R. C., 1989, Solid Phase, Peptide Synthesis, IRL Press].

The compounds of formula (I), which form the subject of the present invention, may be synthesized using the various techniques that are known in the literature to the man skilled in the art. These techniques include solid-phase peptide synthesis, peptide synthesis in solution, synthetic methods of organic chemistry, or else any combination of the above. The pre-selected scheme of synthesis will of course depend upon the composition of the particular molecule. Preferably, synthetic methods based upon appropriate combinations of solid-phase techniques are used [Atherton, E. & Sheppard, R. C., 1989, Solid Phase Peptide Synthesis, IRL Press; Gross, E. & Meinhofer, J., 1980, The Peptides: Analysis, Synthesis and Biology, Vol. 2; Merrifield, B., 1986, Science 232, 341] and the classic in-solution methods [Gross, E. & Meinhofer, J., 1980, The Peptides: Analysis, Synthesis and Biology, Vol. 1], which involve low production costs, in particular on an industrial scale. In detail, these methods may be the ones described in what follows.

Synthesis in solution [Gross, E. & Meinhofer, J., 1979, The Peptides: Analysis, Synthesis and Biology, Vol. 1] of fragments of the peptide chain through the subsequent coupling of N-protected amino acids, appropriately activated, to an amino acid or to a C-protected peptide chain [Gross, E. & Meinhofer, J., 1981, The Peptides: Analysis, Synthesis and Biology, Vol. 3; Gross, E. & Meinhofer, J., 1983, The Peptides: Analysis, Synthesis and Biology, Vol. 5], with isolation of the intermediates, subsequent selective de-protection of the N- and C-terminal ends of said fragments, and coupling thereof until the desired peptide is obtained. Solid-phase synthesis of the peptide chain from the C-terminal end towards the N-terminal end on an insoluble polymeric substrate. For completion of the desired peptide chain, there follows the removal of the peptide from the solid substrate, with simultaneous de-protection of the side chains, by acidic hydrolysis, in the presence of appropriate “scavangers” [Atherton, E. & Sheppard, R. C., 1989, Solid Phase Peptide Synthesis, IRL Press; Gross, E. & Meinhofer, J., 1980, The Peptides: Analysis, Synthesis and Biology, Vol. 2; Merrifield, B., 1986, Science 232, 341].

The synthetic metalloproteins of the present invention may be advantageously used for making biosensors of affinity with great advantages in their use.

In fact, the use of the compounds of formula (I), as essential elements of an electrochemical device, makes it possible to avoid recourse to marked analytes or to complicated intermediate manipulations. The recognition of the analyte by the element of recognition may be directly detected by means of a variation in the redox potential of the pseudo-metalloprotein, to which the element of recognition binds in a covalent or non-covalent way.

According to the present invention, a biosensor is constructed by binding the synthetic metalloproteins which form the object of the present invention to an electrode, through the group X2. The group X1 is capable of recognizing an analyte, and this recognition causes a variation in the redox potential of M. This variation may be measured using electrochemical methods, such as potentiometric or voltmetric methods.

The technology based upon the synthetic metalloproteins of the invention does not involve a cascading of events in the transduction of the signal, which may, instead, be transferred directly, and possibly amplified, to the detection system. This aspect is particularly important because it enables ease of automation and hence high levels of reproducibility, sensitivity and speed of analysis. In addition, the solution proposed for the functionalization of the electrode makes it possible to overcome the problems connected with the orientation of the element of recognition with respect to the electrode and ensures an efficient and fast transfer of the information to the electrode.

When the analyte binds to the element of recognition, there is a variation in the redox potential of the pseudo-metalloprotein. This variation of the redox potential represents a new type of mechanism of transduction of the signal, which enables a direct detection, by means of electrochemical methods, also of active non-redox analytes. The variations in the redox potential may be easily detected and amplified, also by coupling multiple scans of the potential.

An electrode made with the compounds of the invention may be advantageously used for the following purposes: in vitro diagnostics, immunodiagnostics, determination of pollutant substances in water, determination of preservatives in foodstuffs, determination of drugs and/or toxic products in biological fluids, and determination of the metabolic pathways of drugs. In addition, the biosensors comprising the synthetic metalloproteins of the invention may be used as electrochemical probes of biomolecules.

The following examples are given to provide a better illustration of the invention and are not to be considered in any way limiting of the scope thereof.

### EXAMPLE 1

Preparation of the Compound of the General Formula (I) in Which:

M is the Fe³⁺ ion, X1 is the substance P (H-Arg-Pro-Lys-Pro-Gln-Gln-Phe-Phe-Gly-Leu-Met-OH), X2 is Cys, S1 and S2 are Gly-Gly, C1, C2, C3 and C4 are Cys, A1 and B1 are Gln, A2 and B2 are Gln, A3 and B3 are Thr, A4 and B4 are Ile, A5 and B5 are Gly, A6 and B6 are Ala, A7 and B7 are Pro, A8 and B8 are Aib, A9 and B9 are Ser, A10 and B10 are Ile, Y1 and Y3 are Ile, Y2 and Y4 are NH2.

The various phases of the synthesis of the compound Fe³⁺-(Arg-Pro-Lys-Pro-Gln-Gln-Phe-Phe-Gly-Leu-Met-Gly-Gly-Gln-Gln-Cys-Thr-Ile-Cys-Gly-Ala-Pro-Aib-Ser-Ile-Ile-NH₂)(Cys-Gly-Gly-Gln-Gln-Cys-Thr-Ile-Cys-Gly-Ala-Pro-Aib-Ser-Ile-Ile-NH₂) are given in what follows.

### EXAMPLE 1a

Synthesis of the Compound A:

(Seq. 1): Arg-Pro-Lys-Pro-Gln-Gln-Phe-Phe-Gly-Leu-Met-Gly-Gly-Gin-Gin-Cys-Thr-Ile-Cys-Gly-Ala-Pro-Aib-Ser-Ile-Ile-NH₂.

The synthesis of the compound A was performed using the strategy of solid-phase peptide synthesis, and using an automatic peptide synthesizer, which operates in continuous flow. In particular, the methodology was used that employs, as protector group of the alpha-amine function, the Fmoc group [Atherton, E. & Sheppard, R. C., 1989, Solid Phase Peptide Synthesis, IRL Press]. The following protections were used on the side chains: Arg(Pmc), Lys(Boc), Gln(Trt), Glu(OtBu), Ser(tBu), Asn(Trt), Cys(Trt), Thr(tBu). The synthesis was conducted using as solid substrate the resin Novasyn PR 500, which enables the peptide to be obtained as C-terminal amide. A scale of synthesis of 0.2 mmol was adopted, using a substitution of the resin of 0.36 mmol/g. For removal of the Fmoc group from the alpha-amine function of each residue, after the coupling, a 20% solution in volume of piperidine in DMF was used. Two successive treatments, one of 3 minutes and the other of 7 minutes, were used for each cycle. The amino acids were inserted in subsequent stages, and the conditions and methodologies used for each individual residue were the following: all the Fmoc-amino acids, with the exception of Fmoc-Ile, Fmoc-Aib and Fmoc-Thr(tBu), were inserted with the procedure of the PyBop: 4 equivalents of the Fmoc-protected amino acid, 4 equivalents of PyBop and 8 equivalents of DIEA in DMF with an acylation time of two hours [Coste, [symbol] ., Le-Nguyen, D., & Castro, B. (1990) Tetrahedron Left. 31, 205]. Each stage of the coupling was repeated twice. Fmoc-Ile, Fmoc-Aib and Fmoc-Thr(tBu) were introduced using as activating agent HATU [Abdelmoty, I., Albericio, F., Carpino, L. A., Foxman, B. M., Kates, S. A., 1994, Left. Pep. Sci. 1, 57]: 4 equivalents of the Fmoc-protected amino acid, 4 equivalents of HATU and 8 equivalents of DIEA in DMF with an acylation time of two hours. Each stage of the coupling was repeated twice. Controls using the Kaiser test were carried out to evaluate the completeness of each coupling reaction. The detachment of the peptide from the resin and the simultaneous removal of the protector groups of the side chains were carried out using a mixture of ethanedithiol/triisopropylsilane/H2O/TFA in the ratio of 0.25/0.1/0.25/9.4 (in volume) at 0° C. for 3 h. The resin was filtered, and the crude peptide was precipitated from the acidic solution with ethyl ether. The crude product was obtained in the form of a powder, with a yield of 80%, based on the level of substitution of the resin. The crude product was reduced with dithiotreitol, using an excess of 5 times with respect to each SH group present in the amino acid sequence. The reaction was conducted in an aqueous solution at pH 8, for 3 h at 40° C. The homogeneity of the product was obtained from the analysis by analytical HPLC, which showed the presence of a single main peak at a tr=20.5 min. The crude material was purified by means of preparative RP-HPLC to obtain 0.200 g of pure product. Analysis by means of analyticaI HPLC confirmed the purity of the product. The identity of the product was confirmed by means of MALDI-TOF mass spectrometry, which confirmed the expected molecular weight of 2762 uma.

### EXAMPLE 1b

Synthesis of Compound B:

Synthesis of compound B. (Seq. 2): Cys-Gly-Gly-Gln-Gln-Cys-Thr-Ile-Cys-Gly-Ala-Pro-Aib-Ser-Ile-Ile-NH₂.

Synthesis of compound B was conducted using the strategy described for compound A. The crude product was obtained in the form of a powder, with a yield of 85%, based on the level of substitution of the resin. The crude product was reduced with dithiotreitol, using an excess of 5 times with respect to each SH group present in the amino acid sequence. The reaction was conducted in an aqueous solution at pH 8, for 3 h at 40° C. The homogeneity of the product was obtained from analysis by means of analytical HPLC, which revealed the presence of a single main peak at tr=15.5 min. The crude material was purified by means of preparative RP-HPLC to obtain 0.160 g of pure product. Analysis by means of analytical HPLC confirmed the purity of the product. The identity of the product was confirmed by MALDI-TOF mass spectrometry, which confirmed the expected molecular weight of 1535 uma.

### EXAMPLE 2

Synthesis of the compound Fe³⁺-(Arg-Pro-Lys-Pro-Gln-Gln-Phe-Phe-Gly-Leu-Met-Gly-Gly-Gln-Gln-Cys-Thr-Ile-Cys-Gly-Ala-Pro-Aib-Ser-Ile-Ile-NH₂)(Cys-Gly-Gly-Gln-Gln-Cys-Thr-Ile-Cys-Gly-Ala-Pro-Aib-Ser-Ile-Ile-NH₂). For the preparation of this compound, all the operations were performed in strictly anaerobic conditions, and the following procedure was adopted:

Complexation Reaction

An aqueous solution of 2×10⁻³ M of Compound A was prepared.

An aqueous solution of 2×10⁻³ M of Compound B was prepared.

Equal volumes of the two solutions A and B were mixed together, and to the resulting solution was added Fe(SO4)2(NH4)2 in a stoichiometric quantity. The final solution was brought to pH 7.5 by addition of NaOH. The formation of the complex was confirmed by means of uv-vis spectroscopy, for the presence of absorption bands with maxima at 310 and 332 nm [Im, S.-C., & Sykes, A. G., 1996, J. Chem. Soc. Dalton Trans. 2219].

The iron was oxidized to the state of oxidation +3 by means of exposure to air of the solution prepared in stage 3. The solution assumed a red colouring. The formation of the complex was confirmed by uv-vis spectroscopy, for the presence of absorption bands with maxima at 358, 486, 565 nm.

List of Abbreviations

For the nomenclature and the abbreviations of the amino acids, reference is made to the recommendations of the IUPAC-IUB Joint Commission on Biochemical Nomenclature (Eur. J. Biochem. 1984, 138, pp 9); the amino acids are understood as in the L configuration if not otherwise specified. The other abbreviations used are:

Orn=ornithine, Gly=glycine, Ala=alanine, Val=valine, Leu=leucine, delta-Leu=dehydroleucine, alpha-Me-Leu alpha-methylleucine, Ile=isoleucine, Pro=proline, Phe=phenylalanine, Trp=tryptophan, Met=methionine, Ser=serine, Thr=threonine, Tyr=tyrosine, delta-Tyr=alpha-beta-dehydrotyrosine, alpha-Me-Tyr=alpha-methyltyrosine, Asn=asparagine, Gln=glutamine, Asp=aspartic acid, Lys=lysine, His=histidine, Glu=glutamic acid, Arg=arginine, Nle=norleucine, Hyp=hydroxyproline, delta-Pro=dehydroproline, delta-Glu=alpha-beta-dehydroglutamic acid, alpha-Me-Glu=alpha-methyl-glutamic acid, Pgl=phenylglycine, 1-Nal=beta-1-naphthylalanine, 2-Nal=beta-2-naphthylalanine, Cha=cyclohexylalanine. alle=allo-isoleucine, Chg=cyclohexylglycine, Sar=sarcosine, Pip=pipecolic acid, Azt=azetidinic acid, Gla=gamma-carboxyglutamic acid, Pap=para-amidino-phenylalanine, Deg=diethylglycine, Dpg=dipropylglycine, aThr=allo-threonine, Aba=alpha-amino-n-butyrric acid, Pba=aminophenylbutyrric acid, S-Pro=thioproline, Aib=alpha-aminoisobutyrric acid (alpha-methyl-alanine), Dap=2,3 diaminoproprionic acid, Dab=2,4 diaminobutyrric acid, Gaba=gamma-aminobutyrric acid, epsilon-Aca=epsilon-aminocaproic acid, delta-Ava=delta-aminovaleric acid, beta-Ala=beta-alanine, Ac₃c=alpha-aminocyclopropane carboxylic acid, Ac₄c=alpha-amino-cyclobutane carboxylic acid, Ac₅c=alpha-aminocyclopentane carboxylic acid, Ac₆c=alpha-aminocyclohexane carboxylic acid, alpha-Ac₅c=alpha-aminocyclopentane carboxylic acid, alpha-Ac₆c=alpha-aminocyclohexane carboxylic acid, Dph=diphenylglycine, Boc=tert-butyloxycarbonyl, Fmoc=fluorenylmethoxycarbonyl, Bzl=benzylester, PAM=phenylacetoxymethyl, TFA=trifluoro acetic acid, DCM=dichloromethane, DIEA=diisopropylethylamine, DMF=dimethylformamide, OBzl=benzylether, PyBop=benzotriazole-1-yl-oxy-tris-pyrrolidine-phosphonium-hexafluorophosphate, DCC=dicyclohexylcarbodiimide, DCU=dicyclohexylurea, Bom=benzyloxymethyl, Tos=tosyl, HATU=-O-(7-azabenzotriazole)-1,1,3,3-tetramethyluroniohexafluoro phosphate; OtBu=tert-butyl ester; tBU=tert-butyl ether; Pmc=pentamethylchromanesulphonyl; Trt: triphenyl-methyl.

1. A compound of the general formula (I): 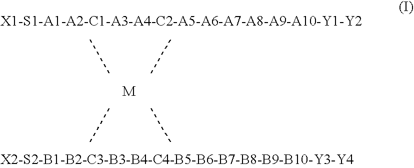
in which:
M is a metal selected in the group made up of Fe, Mn, Ti, Mo, Co, Ni, Cu, Pd, Pt, Au, Ru, Cr, V, Tb, Yb, Rh, Ir, Os;
X1 is an antigen, or else a functional group that enables the association, either covalent or non-covalent, to a biomolecule;
X2 is a functional group that enables the association, either covalent or non-covalent, to an electrode;
S1 and S2, which are the same as or different from one another, are spacer groups which, in the main chain, are made up of 3 to 12 atoms selected in the group of the chemical species C, N, O, S and corresponding mixtures;
C1, C2, C3, C4, which are the same as or different from one another, are natural or synthetic amino acids capable of co-ordinating the metal ion M;
A1, A2, A9, B1, B2, B9, which are the same as or different from one another, are a natural or synthetic hydrophilic amino acid;
A3, A4, A7, A8, A10, B3, B4, B7, B8, B10, which are the same as or different from one another, are a natural or synthetic amino acid;
A5 and B5, which are the same as or different from one another, are Gly or else an amino acid of configuration D on the alpha carbon;
A6 and B6, which are the same as or different from one another, are an amino acid chosen in the group made up of Ala, Abu, Val, Ile, allo-Ile;
Y1 is a natural or synthetic hydrophobic amino acid, and Y2 is Gly, or else Asp, or else Glu, Y3 is a natural or synthetic basic amino acid, and Y4 is NH2; or else Y1 is a natural or synthetic basic amino acid, and Y2 is NH2, Y3 is a natural or synthetic hydrophobic amino acid, and Y4 is Gly, or else Asp, or else Glu; or else Y1 and Y3, which are the same as or different from one another, are a natural or synthetic hydrophobic amino acid, and Y2 and Y4 are NH2.

2. The compound according to claim 1, in which:
M is a metal selected in the group made up of Fe, Mn, Co, Ni, Cu;
X1 is an antigen, or else a functional group that enables the association, either covalent or non-covalent, to a biomolecule;
X2 is a functional group that enables the association, either covalent or non-covalent, to an electrode;
S1 and S2, which are the same as or different from one another, are (Gly)n with n=1-4;
C1, C2, C3, C4, which are the same as or different from one another, are amino acids selected amongst natural or synthetic ones containing on the side chain a functional group capable of co-ordinating the metal ion M;
A1, A2, B1, B2, which are the same as or different from one another, are an amino acid selected in the group made up of Gln, Asn, Thr, allo-Thr, Ser;
A3 and B3, which are the same as or different from one another, are an amino acid selected in the group made up of Ser, Thr, Val, Pro, Ile, allo-Thr, Allo-Ile;
A4 and B4, which are the same as or different from one another, are an amino acid selected in the group made up of Ser, Thr, Val, Pro, Ile, allo-Thr, Allo-Ile, Lys;
A5 and B5, which are the same as or different from one another, are Gly, or else an amino acid of configuration D on the alpha carbon;
A6 and B6, which are the same as or different from one another, are an amino acid in the group made up of Ala, Abu, Val, Ile, allo-Ile;
A7, A10, B7, B10, which are the same as or different from one another, are a natural or synthetic amino acid;
A8 and B8, which are the same as or different from one another, are a C-alpha,alpha-dialkylated amino acid;
A9 and B9, which are the same as or different from one another, are a natural or synthetic hydrophilic amino acid;
Y1 is a natural or synthetic hydrophobic amino acid, and Y2 is Gly, or else Asp, or else Glu, Y3 is a natural or synthetic basic amino acid, and Y4 is NH2; or else Y1 is a natural or synthetic basic amino acid, and Y2 is NH2, Y3 is a natural or synthetic hydrophobic amino acid, and Y4 is Gly, or else Asp, or else Glu; or else Y1 and Y3, which are the same as or different from one another, are a natural or synthetic hydrophobic amino acid, and Y2 and Y4 are NH2.

3. The compound according to claim 1, in which X1 is a functional group selected among amine, sulphidryl, hydroxyl, biotin.

4. The compound according to claim 1, in which X1 is a receptor antagonist.

5. The compound according to claim 1, in which X1 is a receptor agonist.

6. The compound according to claim 1, in which X1 is an amino acid functionalized with biotin.

7. The compound according to claim 1, in which X1 is an enzymatic inhibitor.

8. The compound according to claim 1, in which X1 is a oligonucleotide.

9. The compound according to claim 1, in which X1 is a PNA.

10. The compound according to claim 1, in which X2 is a functional group containing at least one thiol.

11. The compound according to claim 1, in which X2 is a functional group selected between sulphidryl and hydroxyl.

12. The compound according to claim 1, in which the biomolecule is a molecule selected in the group consisting of antibodies, antigens, enzymes, receptors, nucleic acids, peptides, and proteins.

13. The compound according to claim 1, in which the natural amino acid is chosen in the group consisting of: glycine, alanine, valine, leucine, isoleucine, serine, methionine, threonine, phenylalanine, tyrosine, tryptophan, cysteine, proline, histidine, aspartic acid, asparagine, glutamic acid, glutamine, gamma-carboxyglutamic acid, arginine, ornithine, and lysine.

14. The compound according to claim 1, in which the synthetic amino acid is selected in the group consisting of: norleucine, norvaline, alloisoleucine, allothreonine, homoarginine, thioproline, dehydroproline, hydroxyproline, pipecolic acid, azetidinic acid, homoserine, cyclohexylglycine, alpha-amino-n-butyrric acid, cyclohexylalanine, aminophenylbutyrric acid, beta-(1- or 2-naphthyl)alanine, O-alkylated derivatives of serine, threonine and tyrosine, S-alkylated cysteine, S-alkylated homocysteine, epsilon-alkylated lysine, and delta-alkylated ornithine.

15. The compound according to claim 1, in which the hydrophilic amino acid is selected in the group consisting of: Glu, Asp, Asn, Gln, Thr, allo-Thr, h-Ser, Ser, Lys, Arg, His, Orn, Dab, Dap.

16. The compound according to claim 1, in which the hydrophobic amino acid is selected in the group consisting of: Ala, Val, Leu, Ile, allo-Ile, Met, Phe, Tyr, Trp, Cha, Chg, Abu, n-Val, n-Leu, beta-1-Nal, beta-2-Nal.

17. The compound according to claim 1, in which the basic amino acid is selected in the group consisting of: Lys, Arg, His, Om, Dab, Dap.

18. The compound according to claim 1, in which the amino acid capable of co-ordinating metal ions is selected in the group consisting of: Cys, His, Met, Asp, Glu, Lys, Ser, Thr, h-Cys, h-Ser, Dap, Dab, Orn, Gaba.

19. The compound according to claim 2, in which the C-alpha, alpha-dialkylated amino acid is selected in the group consisting of: alpha, alpha-dimethylglycine, alpha-aminocyclopropane carboxylic acid, alpha-aminocyclobutane carboxylic acid, alpha-aminocyclopentane carboxylic acid, alpha-aminocyclohexane carboxylic acid, diethylglycine, dipropylglycine, diphenylglycine.

20. The compound according to claim 1, in ionic form.

21. The compound according to claim 1, bound, through the S atom, to an electrode.

22. Method for the preparation of the compounds according to claim 1, which comprises causing to react in solution fragments of a peptide chain by means of subsequent coupling of N-protected amino acids activated with an amino acid or a C-protected peptide chain, subsequent isolation of the corresponding intermediates, subsequent selective de-protonation of the N- and C-terminal ends of said intermediates and coupling thereof until the desired peptide is obtained.

23. A method for the preparation of the compounds according to claim 1, comprising the reaction of the peptide chain from the C-terminal end towards the N-terminal end on an insoluble polymeric substrate, for completing which there follows the removal of the peptide from the substrate, with simultaneous de-protonation of the side chains, by means of acidic hydrolysis in the presence of a scavenger.

24. An electrochemical biosensor of affinity of a direct type which uses as signal transducers the compounds according to claim 1.

25. The biosensor according to claim 24, obtained by chemical bonding between an electrode and one of the compounds of formula (I) through the group X2.


SEQUENCE LISTING (2 sequences)

SEQ ID NO: 1 - protein, 26 aa, Artificial
  MISC_FEATURE (1)..(26): Aminoacid in position 23 is 2-Aminoisobutyric acid. Peptide is synthetized as in Ex. 1a.
RPKPQQFFGLMGGQQCTICGAPXSII

SEQ ID NO: 2 - protein, 16 aa, Artificial
  MISC_FEATURE (1)..(16): Aminoacid in position 13  is 2-Aminoisobutyric acid Peptide is synthetized as in Ex. 1b.
CGGQQCTICGAPXSII